(19) United States
(12) Patent Application Publication
(10) Pub. No.: US 2016/0242693 A1
(43) Pub. Date: Aug. 25, 2016

(54) PROMOTING PHYSICAL ACTIVITY

(71) Applicant: KONINKLIJKE PHILIPS N.V., Eindhoven (NL)

(72) Inventors: JOSEPH HENRICUS GERARDUS ROUS, EINDHOVEN (NL); LUCIA MANUHUTU, EINDHOVEN (NL)

(21) Appl. No.: 15/029,659

(22) PCT Filed: Oct. 17, 2014
(86) PCT No.: PCT/IB2014/065405

§ 371 Date: Apr. 15, 2016

Related U.S. Application Data
(60) Provisional application No. 61/900,121, filed on Nov. 5, 2013.
(60) Provisional application No. 61/891,962, filed on Oct. 17, 2013.

(51) Int. Cl. A61B 5/00; A61B 5/11; A63B 24/00; H04W 84/18; H04L 29/08

(52) U.S. Cl. CPC A61B 5/486; H04W 84/18; H04L 67/12; A63B 24/0062; A61B 5/1118; A61B 5/6802; A61B 5/6898; A63B 2220/40; A63B 2220/17; A63B 2230/045; A63B 2230/505

(57) ABSTRACT

Methods and systems for promoting physical activity by users utilizing a plurality of components. Embodiments of the present invention collect data concerning user activity, analyze the data, and provide feedback to promote physical activity by the user. These functions may be provided by a single component or dispersed across a plurality of components, and may utilize a variety of intermediary devices to facilitate communications and presentation.

### CROSS-REFERENCE TO RELATED APPLICATIONS

The present application claims the benefit of co-pending U.S. provisional application No. 61/891,962, filed on Oct. 17, 2013, and co-pending U.S. provisional application No. 61/900,121, filed on Nov. 5, 2013; the entire disclosure of both applications are hereby incorporated by reference as if set forth in its entirety herein.

### FIELD

The invention relates generally to methods and apparatus for promoting physical activity, and more specifically to methods and apparatus that encourage physical activity through monitoring and feedback.

### BACKGROUND

Until recently, physical activity was a regular part of life and, also, survival. Many people worked in agriculture and engaged in physical activity on a daily basis, aiding their health and well-being. Limited automobile ownership and compact town and city centers encouraged walking for commuting purposes, for shopping, for deliveries, etc.

The increasing use of labor-saving devices and automation has improved productivity and standards of living, but at a price. Children and adults are increasingly sedentary and their lifestyle choices are having adverse health effects, including the increased prevalence of obesity, cardiac illness, diabetes, etc. While medical science has developed techniques to address some of these symptoms they do not treat the underlying cause, which is the lack of physical exercise.

Accordingly, there is a need for methods and apparatus that encourage individuals, adults and children, to engage in physical activity.

### SUMMARY OF THE INVENTION

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

Various embodiments of the present invention provide a system for encouraging physical activity. The system comprises devices that monitor a user's activity and give feedback to the user concerning their activity level. More typical embodiments include a plurality of devices, with each device performing one or more roles.

For example, one device associated with a user includes one or more sensors for monitoring the user's activity. That same device may give the user immediate feedback concerning their activity level, or it may transmit the data to one or more devices for further processing and subsequent feedback. The device receiving the transmissions may itself process the data concerning the user's activity level or it may act as a relay for the data to yet another device that performs that processing.

The device that ultimately processes the data concerning the user's activity level may provide feedback to the user concerning their activity level or it may transmit the feedback to one or more devices for delivery to the user. For example, the device receiving the feedback may itself deliver the feedback to the user or it may act to relay the feedback to yet another device that ultimately delivers the feedback.

In one aspect, embodiments of the present invention provide an activity monitor. The activity monitor includes a physiological sensor configured to measure data concerning user activity and a transceiver for wireless communications. The transceiver communicates measurements of data concerning user activity to an intermediary device, the intermediary device configured to provide feedback concerning user activity, the feedback concerning user activity selected from the group consisting of changing the appearance of an avatar in response to user activity, changing the set of avatars available for display, and any combination of the foregoing.

In one embodiment, the transceiver is used to determine proximity to a wireless beacon. In one embodiment, the monitor is configured to be worn by a user. In one embodiment, the data concerning user activity is selected from the group consisting of heart rate data, temperature data, and pedometer data. In one embodiment, the feedback concerning user activity is presented in response to the physiological sensor measuring data concerning user activity that indicates the user activity exceeds a specified value.

In another aspect, embodiments of the present invention provide an analytics device for encouraging user activity. The device includes a transceiver for receiving communications comprising data concerning user activity, a processor configured to analyze data concerning user activity, and an interface for presenting feedback from the data analysis to a user via an intermediary device, wherein the feedback from the data analysis is selected from the group consisting of a message changing the appearance of an avatar in response to user activity, a message changing the set of avatars available for display, and any combination for the foregoing.

In one embodiment, the communications comprising data concerning user activity are received from an activity monitor via an intermediary device. In on embodiment, the feedback from data analysis includes an analysis of user location compared against a predetermined route. In one embodiment, the processor analyzes the data concerning user activity and compares the data against a desired level of user activity before presenting feedback from the data analysis. In one embodiment, the feedback is presented on a regular basis.

In still another aspect, embodiments of the present invention provide a method of encouraging user activity. The method includes receiving communications comprising data concerning user activity obtained from a physiological sensor, analyzing the data concerning user activity, and presenting feedback from the data analysis utilizing an intermediary device, wherein the feedback from the data analysis is selected from the group consisting of a message changing the appearance of an avatar in response to user activity, a message changing the set of avatars available for display, and any combination of the foregoing.

In one embodiment, the feedback from the data analysis is presented to a user on a regular basis. In one embodiment, the method further comprises presenting a report of user activity. In one embodiment, the feedback is used to incentivize a user to engage in a certain level of activity. In one embodiment, the feedback concerns the user's satisfaction of a predetermined set of goals.

In yet another aspect, embodiments of the present invention provide an activity monitor. The activity monitor includes a physiological sensor configured to measure data concerning user activity and a transceiver for wireless communications, wherein the transceiver is configured to transmit wireless communications comprising data concerning user activity, the wireless communications used to influence the contents of a story presented to the user of the activity monitor.

In one embodiment, the monitor is configured to be worn by a user. In one embodiment, the wireless communications comprising data concerning user activity are transmitted in response to the physiological sensor measuring data concerning user activity that indicates the user activity exceeds a specified value.

In still another aspect, embodiments of the present invention provide an analytics device for encouraging user activity. The device includes a transceiver for receiving communications comprising data concerning user activity, a processor configured to analyze data concerning user activity, and an interface for providing a story whose contents vary in response to the results of the data analysis.

In one embodiment, the communications comprising data concerning user activity are received from an activity monitor via an intermediary device. In one embodiment, the story is presented to a user via an intermediary device. In one embodiment, the intermediary device is selected from the group consisting of a computer, a smartphone, a tablet, and a gaming console.

In one embodiment, the presentation of the story is contingent on the presence of an intermediary device. In one embodiment, the processor analyzes the data concerning the user activity and compares the data against a desired level of user activity before presenting the story. In one embodiment, the story is chosen based at least in part on the data concerning user activity.

In another aspect, embodiments of the present invention provide a method of encourage user activity. The method includes receiving communications comprising data concerning user activity obtained from a physiological sensor, analyzing the data concerning user activity, and providing a story whose contents vary in response to the results of the data analysis.

In one embodiment, the story is presented to a user utilizing an intermediary device. In one embodiment, the story is only presented when the intermediary device is in communication with a second intermediary device. In one embodiment, the method further comprises processing a purchase transaction story for later presentation. In one embodiment, the story is chosen based at least in part on the data concerning user activity.

These and other features and advantages, which characterize the present non-limiting embodiments, will be apparent from a reading of the following detailed description and a review of the associated drawings. It is to be understood that both the foregoing general description and the following detailed description are explanatory only and are not restrictive of the non-limiting embodiments as claimed.

### BRIEF DESCRIPTION OF DRAWINGS

Non-limiting and non-exhaustive embodiments are described with reference to the following figures in which:

FIG. 1 is a diagram of an exemplary system for encouraging user activity in accord with the present invention;

FIG. 2 is a block diagram of the activity monitor 100 of FIG. 1 in accord with one embodiment of the present invention;

FIG. 3 is a block diagram of one embodiment of the analytics device 104 of FIG. 1 in accord with the present invention;

FIG. 4 is a diagram of another embodiment of the present invention for encouraging user activity; and

FIG. 5 is a flowchart presenting an embodiment of a method for promoting physical activity in accord with the present invention.

In the drawings, like reference characters generally refer to corresponding parts throughout the different views. The drawings are not necessarily to scale, emphasis instead being placed on the principles and concepts of operation.

### DETAILED DESCRIPTION

Various embodiments are described more fully below with reference to the accompanying drawings, which form a part hereof, and which show specific exemplary embodiments. However, embodiments may be implemented in many different forms and should not be construed as limited to the embodiments set forth herein; rather, these embodiments are provided so that this disclosure will be thorough and complete, and will fully convey the scope of the embodiments to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one embodiment of the invention. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are the means used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the present invention include process steps and instructions that could be embodied in software, firmware or hardware, and when embodied in software, could be downloaded to reside on and be operated from different platforms used by a variety of operating systems.

The present invention also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein, and any references below to specific languages are provided for disclosure of enablement and best mode of the present invention.

In addition, the language used in the specification has been principally selected for readability and instructional purposes, and may not have been selected to delineate or circumscribe the inventive subject matter. Accordingly, the disclosure of the present invention is intended to be illustrative, but not limiting, of the scope of the invention, which is set forth in the claims.

In brief overview, embodiments of the present invention encourage users (e.g., adults, children, etc.) to engage in physical activity. Data concerning the user's activity is collected and analyzed, and various forms of feedback are presented in response to that analysis to encourage the user to undertake physical activity. These functions may be provided by a single component or dispersed across a plurality of components, and may utilize a variety of intermediary devices to facilitate communications and presentation.

### Monitoring Activity

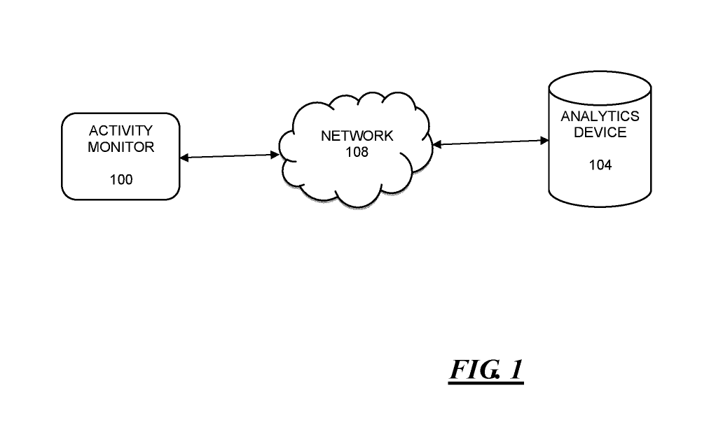

FIG. 1 presents one embodiment of the present invention consisting of an activity monitor 100 that communicates with an analytics device 104 via a communications network 108. The activity monitor 100 collects data concerning a user's activity and transmits it to the analytics device 104 for analysis and follow-up action.

In this embodiment, the follow-up action includes the display of feedback to a user on the activity monitor 100 to encourage the user to undertake physical activity. As discussed below, however, other embodiments of the invention use any number of intermediary devices to convey data to the activity monitor 100 or the analytics device 104, or to provide feedback to the user.

The activity monitor 100 is typically some form of dedicated device intended to measure user activity, such as a bracelet, a toothbrush, a pendant, an orb, etc., although it may take the form of a general purpose computing device such as a desktop computer, a laptop computer, a camera-equipped device, a smartphone, or a tablet, etc. As discussed in greater detail below, the activity monitor 100 should either include or be capable of being equipped with various sensing means to permit relevant measurements to be performed measurement of the user's activity. As discussed below, useful sensors include accelerometers, cardiometers, thermometers, pedometers, cameras, etc.

Various implementations of the analytics device 104 include general purpose computing devices such as a desktop computer, a laptop computer, a smartphone, or a tablet, physical server machines such as a locally-hosted server computer, a remotely-hosted server, locally or remotely hosted clusters of servers, a virtual machine hosted by an on-demand computing service such as ELASTIC COMPUTE CLOUD a.k.a. EC2 offered by AMAZON.COM, INC. of Seattle, Wash., etc.

The network 108 may be a local area network (LAN) implemented using, e.g., Ethernet or Token Ring technologies, or a wide area network (WAN) such as the Internet. In some embodiments, the network may be a personal area network (PAN) implemented using, e.g., Bluetooth, ZigBee, physical contact, or capacitive contact through a user's body to transmit data from the activity monitor 100 to analytics device 104. These types of PANs are discussed in more detail in U.S. Pat. Nos. 8,098,129 and 8,594,568, both issued to the assignee, which are both hereby incorporated by reference in their entirety. The terminology used in the present application may differ from that of the patents incorporated by reference and, accordingly, the terminology in those patents should only be used to interpret the embodiments of the invention utilizing PANs.

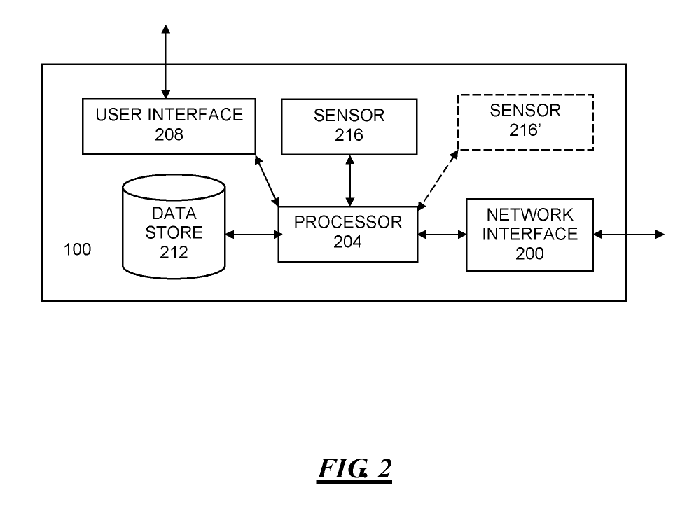

FIG. 2 describes an exemplary activity monitor 100 in additional detail. The activity monitor 100 may be a dedicated device such as a bracelet or an orb containing one or more of the components depicted in FIG. 2 or a general purpose device such as an IPHONE or other smartphone device running a program that collects data concerning user activity as discussed herein.

The network interface 200 allows the activity monitor 100 to transmit communications to other devices such as the analytics device 104 and, in one embodiment, provides an interface to network 108. Suitable network interfaces 200 include gigabit Ethernet, Wi-Fi (802.11a/b/g/n), Bluetooth, and 3G/4G wireless interfaces such as GSM/WCDMA/LTE that enable data transmissions between activity monitor 100 and other devices such as the analytics device 104. A processor 204 generates communications relating to data concerning user activity for transmission through the interface 200 and processes communications received through the interface 200 that originate outside the activity monitor 100. A typical processor 204 is an x86, x86-64, or ARMv7 processor, and the like. The user interface 208 allows the activity monitor 100 to receive commands from and/or provide feedback to the user; one particular form of feedback involves the use of an avatar, such as a cartoon character or a caricature. Exemplary user interfaces include graphical displays, physical keyboards, virtual keyboards, etc. The data store 212 provides both transient and persistent storage for data received via the interface 200, data processed by the processor 204, data received or sent via the user interface 208 and data concerning user activity measured by one or more sensors 216.

The activity monitor 100, in various embodiments, includes one or more sensors 216 that directly or indirectly monitor a user's activity level. For example, an activity monitor 100 shaped like a bracelet or an orb and intended to be worn or carried by a user may include an accelerometer 216 that measures acceleration forces; those measurements may in turn be used by the processor 204 to infer an activity level for the user wearing the bracelet. An activity monitor 100 shaped like an orb that is not intended to be carried by a user may include a camera 216 that can capture sequential images of user; those images may in turn be used by the processor 204 to infer an activity level for the user.

Many different sensing technologies may be used in the activity monitor 100 to collect data that may be used for determinations of user activity level. Suitable sensors 216 include accelerometers (single axis or multi-axis), cameras, cardiometers, positioning sensors (e.g., GPS sensors), thermometers, and pedometers. Any of these sensors or any combination thereof may be used in the activity monitor 100.

As mentioned above, the activity monitor 100 may take a variety of forms. It may appear in a variety of attractive colors, and may be configured to be waterproof, shockproof, and easy to keep track of. The activity monitor 100 may be powered by replacement batteries, such as those employed by a conventional watch, or it may be rechargeable using, e.g., a micro-USB port interface like a tablet or smartphone.

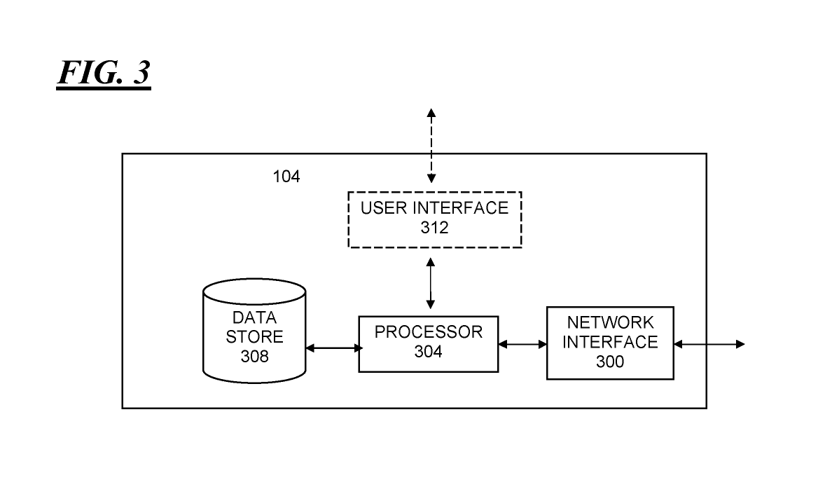

FIG. 3 describes the analytics device 104 in additional detail. As discussed above, the analytics device 104 can take various forms, physical and virtual, but most implementations will share certain common functionalities. The network interface 300 allows the analytics device 104 to receive communications from other devices and, in one embodiment, provides a bidirectional interface to the Internet. Suitable network interfaces 300 include gigabit Ethernet, Wi-Fi (802.11a/b/g/n), and 3G/4G wireless interfaces such as GSM/WCDMA/LTE that enable data transmissions between analytics device 104 and other devices. A processor 304 generates communications for transmission through the interface 300 and processes communications received through the interface 300 that originate outside the analytics device 104. A typical processor 304 is an x86, x86-64, or ARMv7 processor, and the like. The user interface 308 allows the analytics device 104 to receive commands from and/or provide feedback to an operator. Exemplary user interfaces include graphical displays, physical keyboards, virtual keyboards, etc. The data store 312 provides both transient and persistent storage for data received via the interface 300, data processed by the processor 304, and data received or sent via the user interface 308.

The interface 300 of the analytics device 104 is configured to receive communications from various activity monitors 100 and other intermediary devices. The analytics device 104 receives communications from activity monitors 100 such as communications including data concerning user activity or levels of user activity. The analytics device 104 in turn may process the received data to calculate daily activity level targets and compare them against various goals, such as a program specifying a level of activity that may vary with time, location, prior goals achieved, etc. Having processed the received data, the analytics device 104 may communicate with the activity monitors 100 and/or other intermediary devices, sending them communications that are intended to encourage user activity. Such communications may include laudatory messages or images, or messages intended to cause the activity monitor 100 or an intermediary device to display messages or images, such as images of a cartoon avatar that may be, e.g., performing physical activity, among other images.

### Intermediary Devices

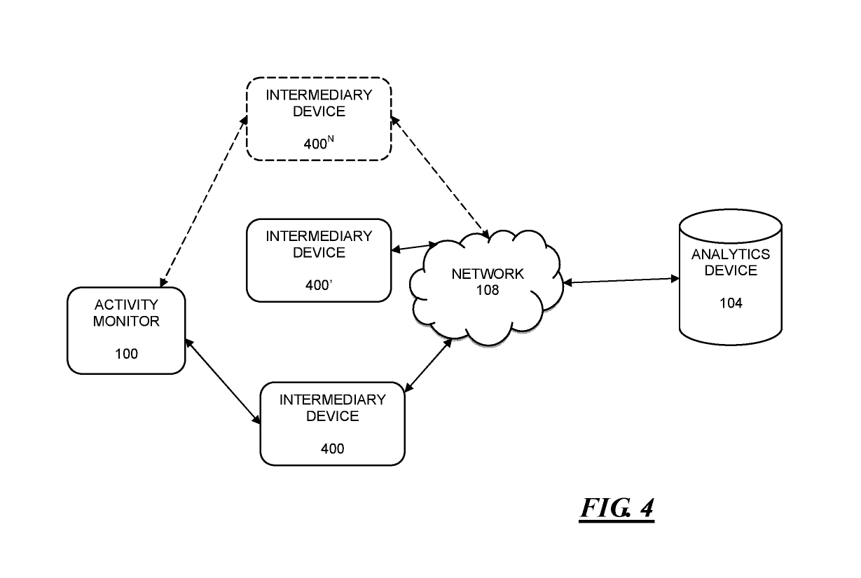

FIG. 4 depicts another embodiment of the present invention. As discussed above, activity monitor 100 collects data concerning user activity and analytics device 104 analyzes the data or derivatives thereof and provides some kind of feedback in response to the analysis, such as recommendations, interventions, or encouragement. In this embodiment, intermediary devices 400 are included in the overall measurement and feedback process.

For example, activity monitor 100 may be equipped with a network interface 200 utilizing BLUETOOTH LOW ENERGY (BLE) technology that is suited for communications with other BLE-equipped devices, but may not provide direct WAN/Internet connectivity. In that case, activity monitor 100 would establish a BLUETOOTH connection with intermediary device 400, e.g., a computer, smartphone, tablet, etc., that is itself in communication with analytics device 104 through network 108. Having established that connection, the activity monitor 100 may then provide data relating to user activity to the analytics device 104 through network 108 by way of intermediary device 400.

In another embodiment, activity monitor 100 may have a limited user interface 208, for example, an LED, a buzzer, or a vibration system. Such a user interface 208 is incapable of delivering, e.g., video or audio feedback, to a user in possession of the activity monitor 100. In this case, the analytics device 104 may operate another intermediary device 400′, such as a phablet, tablet, smartphone, television, electronic billboard, etc., executing an application or otherwise providing feedback to the user by e.g., interacting with a website in communication with the analytics device 104. The intermediary device 400′ may be manually or automatically configured on a per-user basis based on, e.g., its proximity relative to the user possessing the activity monitor 100.

Yet another embodiment may implement the functionality of the analytics device 104 utilizing a plurality of intermediary devices 400. For example, one intermediary device may evaluate the user activity data against a predetermined route or set of actions, while one or more intermediary devices 400 operate as a computing cluster to determine various statistics of interest (e.g., mean, maximum value, minimum value, etc.) from the data relating to user activity.

Still other embodiments may use a plurality of intermediary devices 400 using, e.g., one intermediary device 400 to provide feedback and another intermediary device 400′ in communication with yet another intermediary device 400^{N} to establish communications with the analytics device 104.

There are many reasons that such configurations may prove desirable. In some embodiments, activity monitor 100 may lack one or more of the components depicted in FIG. 2. Such choices can be made for economic or design considerations, such as reducing the size or weight of the monitor 100 or increasing its portability. In other embodiments, activity monitor 100 may include the full suite of components depicted in FIG. 2, but the intermediary devices 400 are utilized for convenience or for other desirable reasons.

For example, one intermediary device 400 in accord with the present invention may be a marble-shaped device (“marble”) that is waterproof, impact resistant, and equipped for wireless communications (e.g., Bluetooth Smart). The marble may be made of semi-transparent plastic and include a light, such as an LED, that may glow when the marble is held or placed on a surface. The marble may be fabricated in two pieces, such that it includes an upper part, housing operative electronics, and a lower part, housing a battery and other components.

Still other intermediary devices 400 may include a vibration element and or a speaker to provide feedback. In other embodiments, the intermediary devices 400 may be a toy or stuffed animal.

In one embodiment the intermediary device 400 is an entertainment item such as storybook (e.g., an e-book) or a video (e.g., a television, a phablet, etc.). The entertainment item may be preloaded with motivational content or configured to load such content on demand. For example, an app or other software installed on a computer, smartphone, tablet, or other computing device may be configured to provide a storybook. The computer, smartphone, tablet, or other computing device is typically configured to be capable of wireless communication (e.g., BLE). The story may be configured to cover a week rhythm or a program rhythm, as discussed in greater detail below.

The user or a third party can purchase content storybooks via the app or other software for display to the user. If the activity monitor 100 or analytics device 104 determines that the user has achieved his daily activity target, the next part of the storybook can be unlocked for display to the user on the intermediary device 400 or the activity monitor 100, e.g., for reading or delivered as an audio book. Also, the level of the user's activity or the activity pattern can be used to alter the presentation of the story by, e.g., introducing new characters, a new story line, etc. The unlocking of additional content may be conditioned on the proximity of the activity monitor 100 to the intermediary device 400, after which the app or other software reacts and shows an unlocking animation on the screen.

Another example of an intermediary device 400 suited for use with embodiments of the present invention is an action-adventure video game with physical toys and a base. The game may include open world creation and story-driven gameplay. Characters, play-sets and other features may be brought into the game using figurines and discs. The base is able to communicate with an activity monitor wirelessly, such as via a Bluetooth Smart communication link. The base may be connected to a game console via a USB connection. In this way the activity monitor 100 can interact via the base and the game console with the video game. Achieving daily, weekly and multi-weekly activity targets can enable power up, new characters, special moves, etc., in the game. The game may be designed to help achieve a multi-week target.

### Encouraging Activity

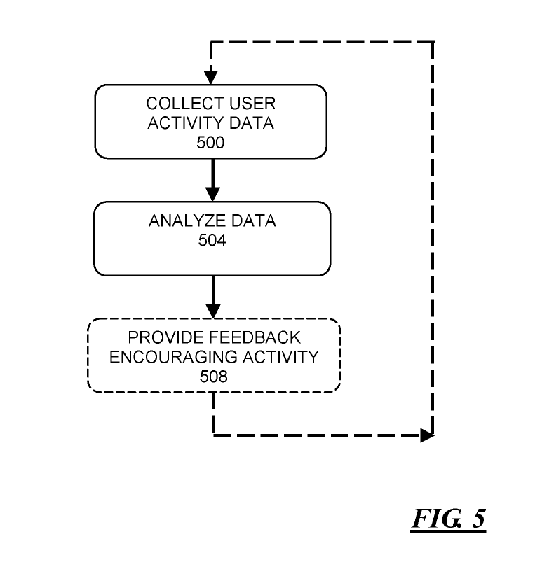

With reference to FIG. 5, one method of the present invention for promoting physical activity utilizes a feedback loop. A device, such as an embodiment of the aforementioned activity monitor, collects data concerning a user's activity (Step 500). The data is communicated, sometimes using one or more intermediary devices, to another device, such as an embodiment of the aforementioned analytics device, which performs various operations on the data (Step 504) and, in view of the operations, provides, optionally using one or more intermediary devices, some kind of feedback (Step 508) to the user intended to encourage physical activity by the user. This process may be executed over a period of days, weeks, months, etc. Some embodiments of the invention provide feedback independent of the received data (e.g., general encouragement or admonishment), while still other embodiments may not provide feedback to a user at all.

The feedback may take a variety of forms in accord with the present invention. In one embodiment, the feedback may consist of a cartoon character, avatar, or other figure whose displayed state varies between at least two states, i.e., active or inactive. The user's activity or level of activity will be monitored using the device, analyzed, and then used to influence the appearance of the figure such that when the user is active the figure will also be displayed as being active. Conversely, when the user is inactive the figure may also be displayed as being inactive. The state of the figure can also assume various intermediate states between activity and inactivity, permitting more nuanced user feedback. The figure may be displayed on the monitoring device itself, or on one or more intermediate devices, such as a smartphone, tablet, television, etc. Other forms of feedback may involve stated goals, images, text, spoken messages, etc.

As mentioned above, the process can be conducted over various and even multiple timeframes. For example, one embodiment of the present invention operates on a daily rhythm. If the user becomes more active each day, as measured by the activity monitor or another device, then appropriate feedback may be provided, such as an avatar being depicted as being more active. Other forms of feedback may include a spoken or written statement encouraging the user to be active enough to give the avatar an “active” or “running” appearance.

Another rhythm, which may be used in another embodiment of the present invention or the same embodiment, independent of the daily rhythm or complementing the daily rhythm, is a weekly rhythm. Each day that a user's activity is sufficient to ensure that the cartoon character avatar gets an “active” or “running” appearance, the avatar will also receive, e.g., a star or other achievement marker. The number of markers may be unlimited or limited to a pre-selected value, e.g., seven stars.

Still another rhythm utilized in various embodiments of the invention, independent of or complementing the other rhythms discussed above, is a program rhythm. This rhythm can be designed to accommodate intermediate or long term activity goals, such as a 12 week fitness program. In order to maintain an increase in user activity over several weeks, the program rhythm can vary the avatars displayed in the daily and weekly rhythms and otherwise provide content, rewards, etc. to leverage the user's interest in collecting digital items or otherwise unlocking achievements.

For example, a new avatar (e.g., a new cartoon character) may be shown if the user, through his activity, causes the current avatar to have a “running” appearance for seven days (or collect seven stars or some other number of achievements). The goal of the overall program may be for the user to unlock all of the avatars potentially available, or otherwise complete a series of achievements or goals through physical activity.

In certain embodiments, each avatar or achievement may be progressively harder to unlock. For example, each new avatar may be more difficult to unlock or bring into a displayed running mode than the previous avatar. Similarly, weeks in a particular program may be progressively more difficult. For example, completing all of achievements in the last week of the program may require significant amounts of user activity. The increased level of difficulty may be offset by an increased reward, such as a special avatar (e.g., a gold colored avatar) or achievement.

As discussed above, one mechanism for encouraging physical activity consists of providing rewards and feedback in various forms to encourage the user to undertake physical activity. Other mechanisms to encourage physical activity used in various embodiments of the invention include the withdrawal of the rewards and positive feedback previously rewarded in response to inactivity. Still other mechanisms include the delivery of negative feedback, such as financial or other penalties.

For example, if a user or their avatar has previously been awarded an achievement (e.g., a star), subsequent inactivity by the user may result in the removal of the achievement. In some embodiments, the failure to make progress, e.g., by unlocking additional avatars or other achievements, may also be treated as equivalent to inactivity and result in the removal of one or more achievements. In some embodiments, the failure to wear the activity monitor for a certain amount of time may also be treated as equivalent to inactivity and result in the removal of one or more achievements, the removal of an unlocked avatar, the reversion to an earlier avatar, etc.

As discussed above, intermediary devices may be used to convey communications between the activity monitor and the analytics device, or otherwise used to provide feedback. The foregoing discussion concerning the encouragement of activity may utilize such an intermediary device in connection with daily rhythms, weekly rhythms, program rhythms, etc.

For example, the marble intermediary device can be used to provide feedback to a user concerning the user's level of physical activity. If the user becomes more active, the marble will, e.g., glow brighter and brighter. If the user reaches his daily activity target, then the marble will, e.g., start to flash (“light party”); the goal will be to make the marble flash every day. This will be a daily rhythm.

There will also be a weekly rhythm. The light party will get more intense each time the daily target is reached. If a user achieves his daily target for one or more days in a row, e.g., (seven days in a row), the marble will change color and the daily rhythm will start again, but in a different color.

The overall program rhythm can require a user to collect a plurality of colors that can be displayed by the marble. For example, a twelve week rhythm can give a user twelve colors to collect. As discussed above, unlocking a new color in a subsequent week may be progressively more difficult to unlock. Making the marble flash in later weeks of the program may require significant levels of activity. If the user's level of activity declines or ceases, then the marble may flash with less intensity or frequency. If the level of inactivity persists for, e.g., several days, then the marble may revert to a previous color.

### Guided Activities

While embodiments of the invention are suited to operate with physical activity of all kinds, some embodiments of the invention involve the use of structured or planned activities to encourage physical activity by a user.

For example, one form of structured activity suited to use with embodiments of the present invention is a game such as a “treasure hunt.” In such embodiments, the activity monitor will typically include some form of wireless communication facility such as BLUETOOTH LOW ENERGY (BLE, and specifically “BLE master” capabilities).

A defined environment, such as a theme park or resort, for example, is configured to contain several “treasures” (i.e., physical objects) or “rally points” (i.e., locations). Each physical object or location includes a low-power transmitter that can notify nearby devices of the transmitter's presence. One such transmitter may be configured to send out IBEACON information that can be received by a smart phone, tablet, or other computing device.

A user equipped with an activity monitor or intermediary device (e.g., a marble, smartphone, etc.) can then wander the defined environment searching for physical objects and locations of interest. The user's device can identify transmitters within its range and assist the user in finding the associated physical object or location by encouraging the user to move in a direction to reduce the distance between the device and the transmitter. If the user's device is brought within a certain range of the transmitter, e.g., three feet or less, then the user's device or another server can update its records to reflect that the user has collected the “treasure” or visited the location of interest.

In one embodiment of the “treasure hunt,” the goal is for the users to visit all of the locations or objects of interest in the defined environment while carrying their device. After reaching this goal, an avatar may be displayed on the user's activity monitor or intermediary device, or other feedback may be provided to the user as discussed above. Because the user will need to exert physical effort as he traverses the defined environment, this may also cause the avatar to have an active appearance, also as discussed above.

In embodiments of the present invention utilizing BLE, the transmitter at the object or location of interest can collect the unique ID number of the user's activity monitor or intermediary device and use this info for data analytics or individualized feedback, such as an e-mail to the user congratulating them on completing the treasure hunt.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

The description and illustration of one or more embodiments provided in this application are not intended to limit or restrict the scope of the present disclosure as claimed in any way. The embodiments, examples, and details provided in this application are considered sufficient to convey possession and enable others to make and use the best mode of the claimed embodiments. The claimed embodiments should not be construed as being limited to any embodiment, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the general inventive concept embodied in this application that do not depart from the broader scope of the claimed embodiments.

What is claimed is:

1. An activity monitor comprising:
a physiological sensor configured to measure data concerning user activity; and
a transceiver for wireless communications,
wherein the transceiver is configured to transmit wireless communications comprising data concerning user activity, the wireless communications used to influence the contents of a story presented to the user of the activity monitor.

2. The activity monitor of claim 1, wherein the monitor is configured to be worn by a user.

3. The activity monitor of claim 1, wherein the wireless communications comprising data concerning user activity are transmitted in response to the physiological sensor measuring data concerning user activity that indicates the user activity exceeds a specified value.

4. A system for encouraging user activity, the device comprising:
a transceiver for receiving communications comprising data concerning user activity;
a processor configured to analyze data concerning user activity; and
an interface for providing a story whose contents vary in response to the results of the data analysis.

5. The system of claim 4, wherein the communications comprising data concerning user activity are received from an activity monitor via an intermediary device.

6. The system of claim 4, wherein the story is presented to a user via an intermediary device.

7. The system of claim 6, wherein the intermediary device is selected from the group consisting of a computer, a smartphone, a tablet, and a gaming console.

8. The system of claim 4, wherein the presentation of the story is contingent on the presence of an intermediary device.

9. The system of claim 4, wherein the processor analyzes the data concerning the user activity and compares such data against a desired level of user activity before presenting the story.

10. The system of claim 4, wherein the story is chosen based at least in part on the data concerning user activity.

11. A method of encouraging user activity, the method comprising:
receiving communications comprising data concerning user activity obtained from a physiological sensor;
analyzing the data concerning user activity; and
providing a story whose contents vary in response to the results of the data analysis.

12. The method of claim 11, wherein the story is presented to a user utilizing an intermediary device.

13. The method of claim 13, wherein the story is only presented when the intermediary device is in communication with a second intermediary device.

14. The method of claim 11, further comprising processing a purchase transaction story for later presentation.

15. The method of claim 11, wherein the story is chosen based at least in part on the data concerning user activity.
